(19) United States
(12) Patent Application Publication
(10) Pub. No.: US 2009/0036565 A1
(43) Pub. Date: Feb. 5, 2009

(54) Dental Composites with a Low Shrinkage Tension and High Flexural Strength

(75) Inventors: Andreas Utterodt, Neu-Anspach (DE); Klaus Ruppert, Maintal (DE); Matthias Schaub, Linsengericht (DE); Christine Diefenbach, Dornburg (DE); Kurt Reischl, Merenberg (DE); Alfred Hohmann, Schmitten (DE); Michael Eck, Schmitten (DE); Nelli Schonhof, Braunfels (DE); Jutta Schneider, Runkel (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS KULZER GMBH, Hanau (DE)

(21) Appl. No.: 12/168,512

(22) Filed: Jul. 7, 2008

(30) Foreign Application Priority Data
Jul. 20, 2007 (DE) 10 2007 034 457.2

(51) Int. Cl. A61K 6/083

(52) U.S. Cl. 523/116

(57) ABSTRACT

Dental composite materials based on (meth)acrylate, exhibiting a proportion of TCD monomers in the total composition of 1-15% by weight and the quotient of flexural strength/shrinkage tension is at least 35.

The invention relates to dental composite materials with a low shrinkage tension and a high flexural strength.

Light-curing materials based on acrylate/methacrylate experience a volume shrinkage during free radical polymerisation as a result of the distance between molecules being reduced during polymerisation and the simultaneous increase in density. This can be substantially reduced by the addition of inorganic fillers such as e.g. dental types of glass or pyrogenic silicic acids since this results in a reduced proportion of monomer per unit of volume and the fillers do not shrink during polymerisation.

In dental applications, the volume shrinkage is of great clinical significance since tensile forces are transferred onto the cavity walls by the material shrinkage. When a maximum force is exceeded, this shrinkage force can, in an extreme case, lead to the cavity wall becoming detached. Bacteria can penetrate into the peripheral gap thus formed and, consequently, secondary caries may arise.

According to DE102005021332A1, light-curing materials based on acrylate/methacrylate have already been presented which exhibit a reduced shrinkage force. This is achieved by various measures: non-agglomerated nanofillers, a mixture of fillers of coarsely and finely particulate dental types of glass, predominant substitution of the highly shrinking diluent TEDMA by UDMA (urethane dimethacrylate), use of tricyclodecane derivatives (in the following abbreviated to TCD) and, optionally, the reduction of the initiator quantity. Only a composition is documented by way of an example therein, and this contains no TCD.

A composite material is produced by intimate mixing of the following components comprising:

| | | | |
|---|---|---|---|
| fillers: | non-agglomerated nanoparticles | 6 | parts by weight |
| | dental glass 1 μm (silanised) | 24 | parts by weight |
| | dental glass 8 μm (silanised) | 53 | parts by weight |
| monomers: | bis-GMA (Bowen) | 11 | parts by weight |
| | UDMA | 4 | parts by weight |
| | TEDMA | 2 | parts by weight |
| initiator(s): | Camphor quinone | 0.1 | parts by weight |
| sum total | | 100.1 | parts by weight |

It is the object of the present invention to provide a composite material for dental applications with a low shrinkage force and a high flexural strength. In particular, the quotient of flexural strength/shrinkage tension is to be optimised.

It has been found that the materials suggested in DE102005021332A1 can be considerably improved. Surprisingly enough it has been found that the ratio of bending strength to shrinkage tension can be increased if a proportion of TCD monomers of 1-15, particularly preferably more than 10% by weight, is present.

The invention consequently relates to

dental composite materials with a total filler content of 70 to 95% by weight containing:
A) in the filler component, 0.5 to 10% by weight of non-agglomerated nanofillers with particle sizes of 1 to 50 nm;
B) in the filler component, at least 60% by weight of a filler mixture of 50 to 90% coarsely and 10 to 50% finely particulate dental types of glass which exhibit a quantitative ratio, based on the average particle size (d₅₀ value) of
finely particulate to coarsely particulate of 1:4 to 1:30,
C) as monomer component, a monomer mixture of
i. 60-80% by weight bis-GMA and a member of the group of TCD-di-HEMA or TCD-di-HEA
ii. 10 to 18% by weight UDMA
iii. the remainder being TEDMA and/or multifunctional crosslinking agents
   D) up to 1% of initiator(s) and
   E) optionally, in the filler component, at least one further dental glass with a particle size which differs from the coarsely and finely particulate dental types of glass.

Non-agglomerated nanofillers are known as such and described e.g. in WO 0130305 A1 or by way of the example of SiO₂ in DE 196 17 931 A1. According to the invention, they preferably belong to the group consisting of: SiO₂, ZrO₂, TiO₂, Al₂O₃ and mixtures of at least two of these substances.

As described in DE 196 17 931 A1 they may be dispersed in organic solvents but also in water or water-containing solvent mixtures.

Suitable as dental types of glass are in particular barium glass powder and/or strontium glass powder. The average particle size of the coarsely particulate dental types of glass is preferably 5-10 μm, in particular approximately 7 μm and that of the finely particulate is 0.5 to 2 μm, in particular 1 μm. Optionally present further dental types of glass have an average grain size of e.g. 2-5 or 10-50 μm.

The filler component may consequently exhibit dental types of glass with a total of three or more grain fractions. It may also contain further conventional fillers common in the dental field such as e.g. quartz mixtures, glass ceramic mixtures or mixtures thereof. In addition, the composites may contain fillers to achieve a high X-ray opacity. The average particle size of the X-ray opaque filler is preferably in the region of 100 to 300 nm, in particular 180 to 300 nm. Suitable as X-ray opaque fillers are e.g. the fluorides of the rare earths described in DE 35 02 594 A1 i.e. the trifluorides of the elements 57 to 71. A filler which is used particularly preferably is ytterbium fluoride, in particular ytterbium trifluoride with an average particle size of approximately 300 nm. The quantity of the X-ray opaque filler preferably amounts to 10 to 50% by weight, particularly preferably 20 to 30% by weight, based on the total filler content.

In addition, precipitated mixed oxides such as e.g. ZrO₂/SiO₂, can be used as fillers. Mixed oxides with a particle size of 200 to 300 nm and in particular approximately 200 nm are preferred. The mixed oxide particles are preferably spherical and exhibit a uniform size. The mixed oxides preferably have a refractive index of 1.52 to 1.55. Precipitated mixed oxides are preferably used in quantities of 25 to 75% by weight, and in particular 40 to 75% by weight.

The fillers are preferably silanised to improve the adhesion between the filler and the organic matrix. Alpha-methacryloxypropyl trimethoxysilane is particularly suitable as adhesion promotor. The quantity of adhesion promoter used depends on the type and the BET surface area of the filler.

In addition, TEDMA and UDMA are suitable for use as multifunctional crosslinking agents: diethylene glycol di(meth)acrylate, decane diol di(meth)acrylate, trimethylol propane tri(meth)acrylate, pentaerythritol tetra(meth)acrylate and butane diol di(meth)acrylate, 1,10-decane diol di(meth)acrylate, 1,12-dodecane diol di(meth)acrylate

To initiate polymerisation, the composites contain a polymerisation initiator, e.g. an initiator for radical polymerisation. Depending on the type of initiator used, the mixtures may be polymerisable cold, by means of light or hot.

The known peroxides such as dibenzoyl peroxide, dilauroyl peroxide, tert.-butyl peroctoate or tert.-butyl perbenzoate can be used as initiators for hot polymerisation, however, alpha,alpha′-azo-bis(isobutyroethyl ester), benzopinacol and 2,2′-dimethyl benzopinacol are also suitable.

Benzoine alkyl ethers or benzoine alkyl esters, benzyl monoketals, acyl phosphine oxides or aliphatic and aromatic 1,2-diketo compounds such as e.g. 2,2-diethoxyacetophenone 9,10-phenanthrene quinone, diacetyl, furile, anisile, 4,4′-dichlorobenzyl and 4,4′-dialkoxybenzyl or camphor quinone are, for example, suitable as photoinitiators. Photoinitiators are preferably used together with a reducing agent. Examples of reducing agents are amines such as aliphatic or aromatic tertiary amines, e.g. N,N-dimethyl-p-toluidine or triethanol amine, cyanoethyl methyl aniline, trimethyl amine, N,N-dimethyl aniline, N-methyl diphenyl amine, N,N-dimethyl sym.-xylidine, N,N-3,5-tetramethyl aniline and 4-dimethylaminobenzoic acid ethyl ester or organic phosphites are examples of reducing agents. Camphor quinone plus ethyl-4-(N,N-dimethyl amino)benzoate, 2-(ethyl hexyl)-4-(N,N-dimethylamino)benzoate or N,N-dimethylaminoethyl methacrylate, for example, are well-established photoinitiator systems.

2,4,6-Tri-methyl benzoyl diphenyl phosphine oxide is particularly suitable as initiator for the polymerisation initiated by UV light. UV-photoinitiators can be used alone, in combination with an initiator for visible light, an initiator for cold curing and/or an initiator for hot curing.

Systems providing radicals, e.g. benzoyl peroxide and/or lauroyl peroxide can be used together with amines such as N,N-dimethyl sym.-xylidine or N,N-dimethyl-p-toluidine as initiators for cold polymerisation.

Dual curing systems, e.g. photoinitiators with amines and peroxides, can also be used.

The initiators are preferably used in quantities of 0.01 to 1% by weight, based on the total mass of the mixture.

During cold polymerisation, it may be appropriate for the composite material to be present divided into two components which are intended to be cured by mixing. It is also possible to provide the material in such a way that it can be cured both by light and by mixing of two components.

Composite materials according to the invention, when used as dental materials, preferably have a quotient of flexural strength/shrinkage tension of >/=35, preferably >/=40, particularly preferably >/=50.

As far as parts or percentages are given these are—as well as in the remaining specification—based on weight unless otherwise indicated.

### EXAMPLES

The results of measurements (Table II) for the mixtures 312, 349, 357, 363, 307 and 206 (comparison, optimised without TCD) which are listed in the following Table I show that the quotient of flexural strength/shrinkage tension increases with a rising TCD content. TCD percentages of more than 10% exhibit values of more than 50.

TABLE I
Formulations

| | Formulation | Formulation | Formulation | Formulation | Formulation | Formulation |
|---|---|---|---|---|---|---|
| Components | 363 | 312 | 357 | 349 | 307 | 206 |
| UDMA | 4.03 | 4.02 | 4.03 | 2.00 | 3.92 | 3.92 |
| Bis-GMA | 10.76 | 9.15 | 9.15 | | | |
| TEGDMA | 1.11 | 1.11 | 1.11 | 2.66 | 0.95 | 0.95 |
| TCDDIHEA | | 1.61 | 1.61 | 10.50 | 12.35 | 12.35 |
| Multifunctional | 0.50 | 0.50 | 0.50 | 0.50 | 1.18 | 1.18 |
| urethane monomer | | | | | | |
| BHT | 0.04 | 0.04 | 0.04 | 0.04 | 0.04 | 0.04 |
| Nano SiO2 (dispersion) | 4.00 | 4.00 | 4.00 | 4.00 | 4.8 | 4.8 |
| Barium aluminosilicate glass | 39.50 | 15.80 | 39.50 | 40.00 | 50.28 | 50.28 |
| filler 0.85μ silanised | | | | | | |
| Barium aluminosilicate glass | | 23.70 | | | 7.66 | 7.66 |
| filler 2μ silanised | | | | | | |
| Barium aluminosilicate glass | 39.50 | 39.50 | 39.50 | 40.00 | 18.53 | 18.53 |
| filler 5μ silanised | | | | | | |
| Light stabiliser 1 | 0.09 | 0.09 | 0.09 | 0.05 | 0.07 | 0.07 |
| Light stabiliser 2 | 0.26 | 0.26 | 0.26 | 0.13 | 0.02 | 0.02 |
| DL camphor quinone | 0.03 | 0.03 | 0.03 | 0.02 | 0.02 | 0.02 |
| Co-initiator | 0.14 | 0.14 | 0.14 | 0.07 | 0.17 | 0.17 |
| PPD | 0.02 | 0.02 | 0.02 | 0.01 | 0.01 | 0.01 |
| Pigments | 0.02 | 0.03 | 0.02 | 0.02 | 0 | 0 |
| Total | 100 | 100 | 100 | 100 | 100 | 100 |

TABLE II
Results of measurements

| | | Modulus of | | | Shrinkage tension | | 3-point flexural | | | |
|---|---|---|---|---|---|---|---|---|---|---|
| | | elasticity in Mpa | | | in Mpa¹ | | strength in Mpa | | | Std. |
| | | | Std.- | TCD | | Std. | flexural | Std. | | deviat. |
| Material | Batch | E-Modul | abw. | monomer | 24 h MW | deviat | strength | deviat. | Quotient | quotient |
| Tetric Evo Ceram (Ivoclar | # G 20087 | 9172 | 308 | | 3.603 | 0.158 | 105.0 | 5.6 | 29.14 | 2.0 |
| Vivadent) | | | | | | | | | | |
| Premise (Kerr) | # 438811 | 7840 | 239 | | 3.679 | 0.143 | 93.0 | 5.5 | 25.28 | 1.8 |
| InTen-S (Ivoclar Vivadent) | # G 02023 | 9506 | 690 | | 3.822 | 0.181 | 109.0 | 11.9 | 28.52 | 3.4 |
| Filtek Supreme XT (3M | # 4 AP | 6528 | 374 | | 4.228 | 0.336 | 111.0 | 10.6 | 26.25 | 3.3 |
| Espe) | | | | | | | | | | |
| EsthetX (Dentsply) | # 305000182 | 11606 | 585 | | 4.388 | 0.240 | 109.0 | 13.6 | 24.84 | 3.4 |
| Herculite XRV (Kerr) | # 06-1262 | 9585 | 305 | | 4.636 | 0.124 | 138.0 | 11.9 | 29.77 | 2.7 |
| Filtek Z250 (3M Espe) | # 4 LPJ | 12174 | 420 | | 4.887 | 0.289 | 153.0 | 15.3 | 31.31 | 3.6 |
| Spectrum TPH (Dentsply) | # 0612001961 | 10367 | 334 | | 4.900 | 0.250 | 127.0 | 10.5 | 25.92 | 2.5 |
| Xtra Fil (VOCO) | # 530835 | 16377 | 669 | | 4.912 | 0.212 | 132.0 | 8.0 | 26.87 | 2.0 |
| Quixfil (Dentsply) | # 0404000401 | 16257 | 783 | | 5.041 | 0.126 | 130.0 | 10.5 | 25.79 | 2.2 |
| Venus (Heraeus) | # 010119 | 9160 | 539 | | 5.180 | 0.252 | 120.0 | 9.6 | 23.17 | 2.2 |
| TPH³ (Dentsply) | # 0409171 | 9804 | 330 | | 5.515 | 0.167 | 128.0 | 13.0 | 23.21 | 2.5 |
| Grandio (VOCO) | # 441238 | 16498 | 544 | | 5.686 | 0.202 | 136.0 | 13.3 | 23.92 | 2.5 |
| Comparative | | 13658 | 548 | 0 | 3.845 | 0.193 | 149.0 | 6.2 | 38.75 | 2.5 |
| example 363 | | | | | | | | | | |
| Example 312 | | 15767 | 560 | 1.61 | 3.250 | 0.087 | 130.0 | 10.4 | 40.00 | 3.4 |
| Example 357 | | 13727 | 272 | 1.61 | 4.060 | 0.185 | 145.0 | 9.4 | 35.71 | 2.8 |
| Beispiel 349 | | 14262 | 661 | 10.5 | 3.330 | 0.143 | 171.0 | 7.6 | 51.35 | 3.2 |
| Example 206 | | 12645 | 559 | 12.35 | 2.796 | 0.119 | 169.0 | 5.0 | 60.44 | 3.1 |
| Examplel 307 | | 13836 | 272 | 12.35 | 3.269 | 0.189 | 172.0 | 11.0 | 52.62 | 4.5 |

¹measured according to the Bonded disc method - Dental Materials (2004) 20, 88-95)

1. Dental composite materials having a total filler content of 70 to 95% by weight and comprising:
a) 0.5 to 10% by weight of non-agglomerated nanofillers with particle sizes of 1 to 50 nm in a filler component;
b) particulate dental glass, at least 60% by weight of a filler mixture of 50 to 90% coarsely and 10 to 50% finely particulate dental types of glass in a ratio, of finely particulate to coarsely particulate of 1:4 to 1:30 in the filler component;
c) as monomer component, a monomer mixture comprising
i. 60-80% by weight bis-GMA and a member of the group of TCD-di-HEMA or TCD-di-HEA
ii. 10 to 18% by weight UDMA
iii. the remainder being TEDMA and/or multifunctional crosslinking agents;
d) up to 1% by weight of initiator(s) and
e) optionally, in the filler component, at least one further dental glass with a particle size which differs from the coarsely and finely particulate dental types of glass; wherein the proportion of TCD monomers in the total composition is 1-15% by weight and a quotient of flexural strength/shrinkage tension is >/=35.

2. Dental composite materials according to claim 1, wherein the proportion of TCD monomers in the total composition is 10-15% by weight.

3. Dental composite materials according to claim 1, wherein the quotient of flexural strength/shrinkage tension is >/=40.

4. Dental composite materials according to claim 1, wherein the quotient of flexural strength/shrinkage tension is >/=50.
